(19) United States
(12) Patent Application Publication
(10) Pub. No.: US 2025/0349406 A1
(43) Pub. Date: Nov. 13, 2025

(54) INTELLIGENT HEALTHCARE FACILITY OPERATIONS TRACKING AND RECORD GENERATING SYSTEM

(71) Applicant: HealthTrek Technologies, LLC, Boca Raton, FL (US)

(72) Inventor: Michael Fagien, Parkland, FL (US)

(21) Appl. No.: 19/203,095

(22) Filed: May 8, 2025

Related U.S. Application Data
(60) Provisional application No. 63/644,746, filed on May 9, 2024.

(51) Int. Cl. G16H 15/00; G16H 10/60

(52) U.S. Cl. CPC G16H 15/00; G16H 10/60

(57) ABSTRACT

Described is a method, comprising: receiving, a template electronic report configured to display information in a graphical format associated with one or more preferences of a physician; receiving, patient information associated with a medical assessment conducted on a patient of the physician; generating a summary electronic report by populating the template electronic report at least in part by processing the patient information and the one or more preferences of the physician with a trained machine learning model; wherein the summary electronic report is configured to display a summary of the medical assessment in the graphical format associated with the one or more preferences of the physician; wherein the summary of the medical assessment comprises a subset of information comprised within the complete electronic record of the medical assessment; and wherein the subset of information comprises at least one image.

### CROSS-REFERENCE

This application claims the benefit of, and priority to U.S. Provisional Application 63/644,746, filed May 9, 2024, and incorporates its disclosure herein by reference in its entirety.

### TECHNICAL FIELD

The subject matter described herein generally relates to medical information systems, and more particularly, to information technology infrastructure configured to interoperate with and supplement radiology information systems (RIS).

### BACKGROUND

Radiology centers often use outmoded information technology (IT) systems (e.g., radiology equipment and software) which complicate effectively conducting day-to-day electronic operations (e.g., scheduling, billing, payroll examination performance tracking, data collection, or data storage). Outmoded IT systems also may provide suboptimal delivery of quality diagnostic exams. These businesses are often slow to incorporate emerging technologies and procedures to improve operations. Increasing demand for radiological scans may compound these problems, increasing error rates and degrading efficiency. Hence, use of legacy IT systems may produce an increased workload for medical staff, resulting in stress, frustration, lower productivity, staff shortages, high turnover, and frustrated employees. This, in turn, may lead to delays or errors in reporting results of radiological assessments, causing dissatisfaction from health care providers (HCPs), patients, and other stakeholders. Even radiology centers equipped with the most advanced IT systems find it difficult to maintain quality customer service and precise reporting as radiological scan volume increases. Further, these centers may also have difficulty integrating continuing medical education (CME), a critical requirement in the rapidly-advancing and high-demand field of radiology, CME is especially important to ensure appropriate use of diagnostic testing. And even the best-performing radiology information systems (RIS) may not adequately address continuous quality improvement (CQI) and CME. These needs become even more difficult to fulfill when adequately trained medical personnel are scarce.

### SUMMARY

In some example embodiments, there may be provided a method including receiving, a template electronic report configured to display information in a graphical format associated with one or more preferences of a physician; receiving patient information associated with a medical assessment conducted on a patient of the physician; and generating a summary electronic report by populating the template electronic report at least in part by processing the patient information and the one or more preferences of the physician with a trained machine learning model. The summary electronic report is configured to display a summary of the medical assessment in the graphical format associated with the one or more preferences of the physician. The summary of the medical assessment comprises a subset of information comprised within a complete electronic record of the medical assessment. The subset of information comprises at least one image.

In some variations, one or more of the features disclosed herein including the following features can optionally be included in any feasible combination. The method further comprises generating a score for a radiological facility based at least in part on the summary of the medical assessment. The patient information comprises a note generated by the physician. The note is a handwritten record. The summary electronic report comprises one or more vital statistics. The summary electronic report comprises one or more treatments or medications administered to the patient during the medical assessment. The summary electronic report comprises demographic or medical information of the patient. The trained machine learning model comprises a natural language processing model. The trained machine learning model comprises a transformer. The trained machine learning model comprises a generative pre-trained transformer model. The trained machine learning model comprises a neural network. The medical assessment is a radiological assessment. The at least one image is a radiological image. The summary electronic report comprises a visual code configured to, when digitally scanned, access a complete electronic record of the medical assessment. The visual code is a quick reference (QR) code.

In some example embodiments, there may be provided a system including a radiology information system (RIS) configured to be implemented by one or more processors, an image server configured to be implemented by one or more processors, and a client device, configured to be implemented by one or more processors. The client device is configured to provide, to the RIS, a template electronic report configured to display information in a graphical format associated with one or more preferences of a physician to the RIS and patient information associated with a medical assessment. The image server is configured to provide, to the RIS, a radiological image. The RIS is configured to generate a summary electronic report by populating the template electronic report at least in part by processing the patient information and the one or more preferences of the physician with a trained machine learning model. The summary electronic report is configured to display a summary of the medical assessment in the graphical format associated with the one or more preferences of the physician. The summary of the medical assessment comprises a subset of information comprised within a complete electronic record of the medical assessment. The subset of information comprises the radiological image.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates a intelligent healthcare facility operations tracking and record generating system, in accordance with some embodiments;

FIG. 2 illustrates a process flow diagram, in accordance with some embodiments;

FIG. 3 illustrates a summary electronic report, in accordance with some embodiments;

FIG. 4 illustrates a computer system;

FIG. 5 is a diagram of an implementation of a transformer model;

FIG. 6A illustrates a set of qualities of radiology centers that a radiology center score is configured to assess;

FIG. 6B illustrates a set of factors that influence the score;

FIG. 6C illustrates a user journey during which metrics for a radiology center are collected and then used to generate the score;

FIG. 7 illustrates an additional embodiment of the intelligent healthcare facility operations tracking and record generating system;

FIG. 8 illustrates an overview of events tracked by a CRM server, in accordance with an embodiment;

FIG. 9 describes events that are tracked by the CRM server related to collecting data on HCPs;

FIG. 10 describes events that are tracked by the CRM server related to visits to an HCP office;

FIG. 11 describes events that are tracked by the CRM server related to order processing;

FIG. 12 describes events that are tracked by the CRM server related to patient arrival. A text and/or phone call is sent to patient morning of exam;

FIG. 13 describes events that are tracked by the CRM server related to conduct of assessments;

FIG. 14 describes events that are tracked by the CRM server related to generation of electronic reports; and

FIG. 15 describes events that are tracked by the CRM server related to center level credentials, in accordance with some embodiments.

### DETAILED DESCRIPTION

Described is an intelligent healthcare facility operations tracking and record generating system improving information technology (IT) infrastructure and incentivizing best practices in radiology centers. A system combining features of a customer relationship management (CRM) platform and a radiology information system (RIS) standardizes care for patients, generates actionable electronic reports for physicians, and provides an objective methodology for assessing the quality of a radiological center.

The described system improves aggregation of relevant clinical data, appropriate use, timely scheduling, exam performance and assessment with rapid delivery of results to healthcare providers (HCPs, e.g., ordering physicians) and their patients. Communication preferences (e.g., methods of communication, vernacular or nomenclature) of HCPs are stored in a server. When a medical assessment is conducted, a machine learning model processes data (e.g., image and/or text data) of the assessment, as well as data collected during a “patient journey,” to generate a summary report configured to adhere to the preferences of the HCP (e.g., an ordering physician). The electronic report may comprise key images from the assessment, selected by the model, to convey the information most prioritized by the HCP. The electronic report may also comprise a visual code (e.g., a quick reference (QR) code) linking to the full assessment and link anatomic descriptions of text to the correlative medical images. The report may convey information in a report in a format or manner most helpful to assist the HCP in the management of patients.

Also, the described system provides an objective, repeatable process for scoring radiological centers. Computing infrastructure may be used to track various metrics associated with a radiological center, which may be related to assessments, patient care, billing, continuing medical education (CME), or other topics. By generating a weighted index incorporating values associated with these metrics, the system may assess the quality of a radiological center, which may be provided as feedback to improve practices and procedures throughout the center and assess productivity of healthcare staff.

Beyond improving efficiency and reducing costs in a radiological center, the described system may effectuate wide-ranging and systematic improvements within the radiology center. The system may guide the actions of radiology center staff (e.g., administrators, technicians, and physicians) to provide better quality patient care and a better quality patient experience, by using data collected about the radiology center and its staff to implement a set of practices and procedures most appropriate for the facility. Because the system can quantify measurable benchmarks, the score may be used as objective evidence of the imaging center's competitive strength.

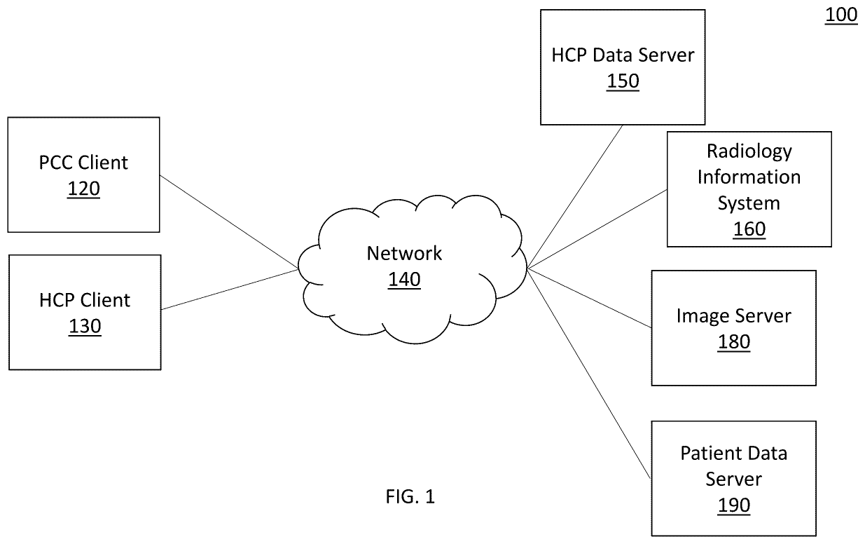

FIG. 1 illustrates intelligent healthcare facility operations tracking and record generating system 100, in accordance with some embodiments. FIG. 1 includes a patient care coordinator (PCC) client 120, a health care provider (HCP) client 130, a network 140, an HCP data server 150, a radiology information system (RIS) 160, an image server 180, and a patient data server 190. Other embodiments of the system may comprise more or fewer components. Additionally, the modular components may be located on a single machine, a distributed computing system, or within another type of computing environment.

The PCC client 120 and the HCP client 130 may be computing devices configured to enable PCCs and HCPs to perform respective electronic services associated with one or more radiological facilities. For example, the PCC client 120 may enable a PCC to enter data related to continuing medical education or provide data about an HCP to HCP data server 150. The HCP client may allow an HCP to generate or view electronic reports (e.g., generate a template electronic report that is configured to provide the HCP with preferred information in a preferred format or configuration), review patient schedules, or provide other services. A client device may be a laptop, a desktop computer, a smartphone, a tablet computer, a personal digital assistant (PDA), a smartwatch, a mainframe computer, or another type of computing device.

Network 140 may comprise hardware and software configured to allow the intelligent healthcare facility operations tracking and record generating system 100 computing devices to communicate with one another. Network 140 may comprise a wired network or a wireless network (e.g., a Wi-Fi network). Network 140 may comprise a local area network (LAN), a wide area network (WAN), or another type of network. In some implementations, the computing devices do not communicate over a network (e.g., when the modular components are located on a single machine, or when storage devices are used to transfer information between different machines housing the modular components).

Radiology Information System (RIS) 160 may coordinate electronic management of images distributed within or provided by the radiology center. RIS 160 may provide patient scheduling, resource management, examination performance tracking, reporting, results distribution, and procedure billing. For example, RIS 160 may support booking appointments, generating reports, handling of documents and electronic reports, billing, and handling workflow within a radiology center.

RIS 160 may comprise one or more software applications. A software application may enable an ordering physician to order exams, schedule assessments with patients, easily communicate with radiology center staff, allow an HCP (e.g., an ordering physician) to submit questions, and deliver electronic reports to the HCP. A software application may also provide an HCP with access to continuing medical education (CME) programs. RIS 160 may use information from HCP data server 150, image server 180, and/or patient data server 190 to generate electronic reports of assessments of patients conducted at a radiology center.

RIS 160 may comprise an electronic ticketing system. The electronic ticketing system may comprise one or more software programs used to process, manage, and track issues (e.g., for patients or HCPs) from submission to resolution. The ticketing system may comprise software for automatically organizing and prioritizing support requests in a central dashboard where radiology center users can tag, categorize, and assign tickets as they come in.

The ticketing system may have access to data from patient data server 190, HCP data server 150, and/or image server 180, to generate a knowledge base including relevant information about users helpful in diagnosing and/or solving their issues and resolutions to common problems faced by users. A ticket may comprise a description of an issue, an urgency level (or deadline for response), and/or a user associated with the issue. Tickets may be resolved by technicians that are on-site at a radiology center or located remotely.

A ticket for a patient may relate to an aspect of the patient's “journey” through a radiology center. For example, each individual step in a protocol for assessing a patient (e.g., scheduling an appointment, reminding a patient of an appointment, greeting a patient, sending a patient to a proper location, or providing a patient with access to data) may generate a ticket. A ticket may be handled by appropriate staff (e.g., the receptionist who greets the patient or the physician who conducts the assessment). A ticket for a physician may relate to a process needed to be completed as part of a patient assessment or may relate to the generation of an electronic report.

Patient data server 190 may store data associated with one or more patients undergoing assessments at the radiology center, such as demographic information and personal information. For example, patient data server 190 may store information that may or may not be necessarily related to a medical assessment of a patient, such as age, residence, sex, height, weight, blood type, medical history, race, marital status, address, or insurance information.

HCP data server 150 may store data related to an HCP (e.g., an ordering physician (OP)). For example, HCP data server 150 may store names, addresses, and contact information for OP contacts, correspondences and expenditures for each OP office (e.g., from front desk to referral coordinator to physician), and details regarding OP preferences for delivery of communications (e.g., electronic correspondence, via a medium such as fax, short message service (SMS), email, WhatsApp, or phone call) and electronic reports, personal information such as spouses' and children's names, birthdays, and hobbies, continuing medical education (CME) and documentation of things delivered to the OP's office. HCP data server may be updated by personnel affiliated with a radiology center or with a healthcare IT system, such as patient care coordinators (PCCs), technologists, or other staff.

Image server 180 may comprise or be incorporated into a picture archiving and communication system (PACS) and may provide storage and access to images from various types of medical imaging devices. Image server 180 may digitally transmit electronic images (e.g., in Digital Imaging and Communications in Medicine (DICOM) format) and electronic reports. The image server 180 may also store, transfer, and manage non-image data (e.g., scanned documents) using industry standard formats, such as Portable Document Format (PDF), once encapsulated in DICOM. Image server may comprise a plurality of imaging modalities, such as X-ray plain film (PF), computed tomography (CT), and magnetic resonance imaging (MRI). Image server 180 may be configured to transmit patient information (e.g., images) via a network (e.g., network 140) or via a storage medium (e.g., a thumb drive). Image server 180 may provide functionality for images to be retrieved and/or reviewed (e.g., on image server 180 itself or on another computing device, such as a remote computing device), and may provide storage for images and reports. Image server 180 may be configured to process images from various medical imaging instruments, including, but not limited to, ultrasound (US), magnetic resonance (MR), Nuclear Medicine imaging, positron emission tomography (PET), computed tomography (CT), endoscopy (ES), mammograms (MG), digital radiography (DR), phosphor plate radiography, visible light photography (VL), histopathology, ophthalmology, or other types of medical images.

Intelligent healthcare facility operations tracking and record generating system 100 may comprise technology that safely maintains a real-time database of essential information with instantaneous permission-level access for designated employees, radiologists, patients, ordering physicians and partners. This access information may relate to who, what, how, where and when data was generated. The technology may comprise cloud technology. The real-time database may comprise a server, or may be co-located with at least one of patient data server 190, HCP data server 150, and/or image server 180. Permissions may limit patient access to data from other patients, or to particular items related to their own assessments. Permissions may limit non-physicians from accessing certain aspects of patient assessments, or sensitive patient information (e.g., personal information). But physicians may be able to have access to sensitive patient information, for the purpose of delivering quality care and for better information patients about assessment results.

The permissions database may be used in conjunction with the electronic ticketing system to facilitate electronic management of patient care. For example, tickets may be generated to be fulfilled only by personnel with appropriate permissions. And in some cases, personnel-issued tickets may automatically be granted (e.g., temporarily or permanently) access to information necessary to fulfill the tickets. For example, a physician may be provided with patient assessment results for the purpose of generating a report.

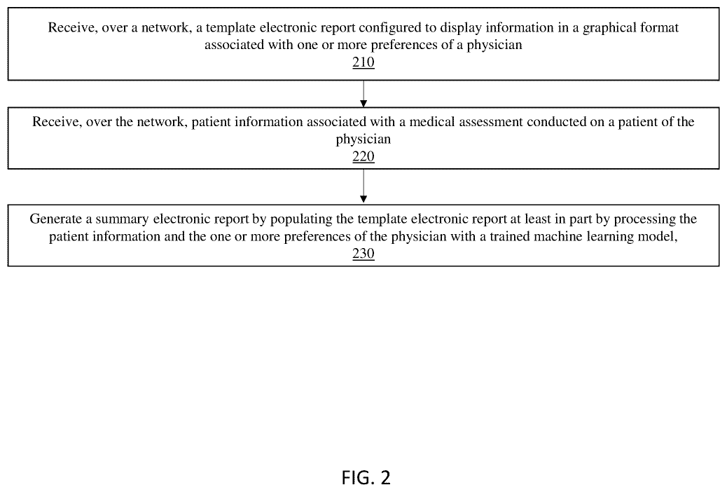

FIG. 2 illustrates a process flow diagram 200, in accordance with some embodiments.

In a first operation 210, a radiology information system (RIS) receives a template electronic report configured to display information in a graphical format associated with one or more preferences of an HCP. For example, the template report may designate that specific areas or sections of the report comprise information relating to particular subject matter (e.g., diagnoses or other test results, patient images, patient demographic or medical information, addresses, dates, or names of personnel). The template report may specify an order in which information is presented. The template report may specify data types to be presented (e.g., images, video, text, or other media). The template report may be configured to be viewable on a device ascertained to be regularly used by the HCP (e.g., viewable on a smartphone, a tablet computer, or a desktop computer). The template report may be configured to display a purpose of the assessment (e.g., diagnosis of a patient, for research, or for teaching).

The preferences of the HCP may be retrieved from a server (e.g., HCP data server 150). The preferences may be entered manually by the HCP or by other personnel. For example, a patient care coordinator (PCC) may visit an HCP office and collect data about HCP communication preferences, and may then store them in the server.

In a second operation 220, the RIS receives patient information associated with a medical assessment conducted on a patient. The patient information may comprise images and/or text describing the type of assessment performed, the date and time of the assessment, one or more images generated from the assessment, and a conclusion of the assessment. The patient information may comprise a set of full results of the assessment. The patient information may also comprise personal information (e.g., age, height, weight, or vital statistics), demographic information (e.g., residence, race, ethnicity, gender, or sex), medical history (e.g., family medical history), or other medical information.

In a third operation 230, the RIS may generate a summary electronic report by populating the template electronic report by processing the patient information and the one or more preferences of the physician with a trained machine learning model.

The machine learning model may comprise a natural language processing (NLP) model and/or a natural language understanding (NLU) model, either or both of which may be based on a transformer architecture. For example, the machine learning model may comprise a generative pre-trained transformer (GPT) model. The machine learning model may also comprise one or more machine learning algorithms, such as neural networks (e.g., convolutional and/or recurrent neural networks), decision trees (e.g., gradient boosted trees or random forests), clustering algorithms (e.g., k-means clustering), or other learning algorithms.

The summary electronic report may include a subset of information from an assessment of a patient. The summary electronic report can include one or more treatments or medications administered to the patient during the medical assessment. The subset may be configured to display information according to the preferences of the HCP. For example, the subset may comprise important demographic and/or medical information about the patient, a diagnosis, and a plurality of key images indicative of the study results. The information may be displayed using vernacular or nomenclature preferred by the patient, determined from the machine learning analysis. The summary electronic report may be configured to omit information determined to be of little importance to the HCP.

The RIS can generate a visual code (e.g., a one-dimensional (1D), two-dimensional (2D) or three-dimensional (3D) visual code) comprising a set of visual patterns and embed it into the summary electronic report. The RIS can encode into the set of visual patterns a link (e.g., a hyperlink) to a location (e.g., locally on the computing device or accessible over a network) where the full assessment is stored. When the visual code is visually or optically scanned by a computing device (e.g., using a camera such as a webcam), the computing device may interpret one or more visual patterns of the visual code to extract the hyperlink and navigate to the stored full assessment. The visual code may be a quick reference (QR) code, a bar code, or another type of visual code.

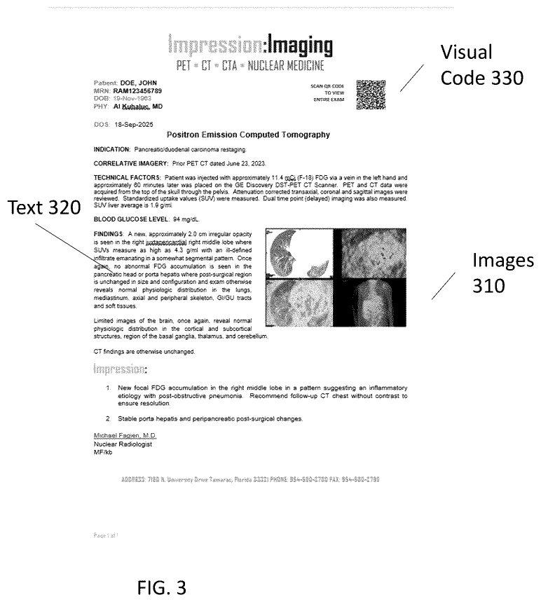

FIG. 3 illustrates a summary electronic report 300, in accordance with some embodiments. The electronic report 300 may comprise text 320, images 310 (e.g., radiological images), or other media associated with a radiological assessment conducted by a physician. The summary electronic report 300 may comprise visual code 330 (e.g., a QR code) that, when scanned, provides a hyperlink configured to allow access to a full version of the performed radiological assessment.

In some embodiments, the summary electronic report 300 may comprise a prioritized worklist comprising one or more priority (e.g., STAT) levels associated with how quickly the report must be generated after an assessment is completed. Example priority levels may comprise STATs 1-4. STAT 1 may correspond to a report that needs to be read as soon as the assessment is completed and hence needs to be generated immediately. STAT 2 may correspond to a report that needs to be generated before the end of the day (e.g., by close of business). STAT 3 may correspond to a report that needs to be generated before the next morning. STAT 4 may correspond with a summary electronic report that may have been initially classified as routine, but has been subsequently modified by office staff or by an HCP (e.g., an ordering physician (OP)).

An example electronic report may include information necessary to inform an HCP (e.g., a radiologist) or other professional of a patient's condition, such as a concise patient history, symptoms or diagnosis gleaned from clinic notes (e.g., typed or handwritten by a physician or other HCP), and a checklist or other object drawing attention to important report or image features (e.g., image annotations such as segmentations or highlighting of organs). Text items of the electronic report may be linked to corresponding images or portions of images. The electronic report 300 may include information written in the professional's desired vernacular and/or nomenclature and may be formatted or structured with respect to a preferred mode of viewing (e.g., via a smartphone or tablet). The report may comprise supplementary medical information or education required to understand the assessment and may describe machine learning or artificial intelligence models used to process the assessment to generate the summary electronic report 300. The summary electronic report 300 may also include a set of “key images,” or images important to conveying the severity of a patient's condition and/or a priority of the scan. These key images may be selected by a machine learning model that processes input data including the preferences of the physician and a natural language understanding of the full assessment. Also, the report may display whether it has been peer-reviewed (e.g., by another physician or HCP).

In some embodiments, a summary electronic report may include some or all of the following information: equipment, supplies, contrast, radiopharmaceutical or other medical products necessary to administer the assessment, a board certification of a radiologist conducting the assessment or a subspecialty expertise and/or curriculum vitae of a radiologist, whether a phone consultation was taken, whether CME was provided and whether or not credits were awarded to medical personnel, an indication whether the assessment was conducted for research or entered as for an exemplary or teaching purpose, radiation safety measures undertaken, incident reporting, peer review information, and report turnaround time information.

In some embodiments, the report may comprise a link to enter a web chat to provide immediate feedback. The electronic report may be provided to a database for outcomes analysis, deep learning using the information in the report, and/or further research. The HCP may approve patient access to the report.

In some cases, a patient may be able to use an artificial intelligence (AI) chatbot to obtain information about the patient's assessment. A machine learning model may process the text of the assessment or the summary electronic report and generate one or more representations (e.g., vectorizations) of the report's context. In this way, the model may determine the “context” or “meaning” of the report, and hence be able to generate a version of the report, or selected text or images from the report, that is accessible or digestible by a lay person.

### Scoring

In some embodiments, the intelligent healthcare facility operations tracking and record generating system may produce a score associated with a radiology facility to denote an effectiveness of the facility's information technology infrastructure (for example, with respect to patient care and/or management operations).

The score may be associated with a set of automatically generated, collected, or tracked objective metrics associated with operations of a radiology center or facility. The metrics may be associated with an HCP, radiology center staff, and/or patients, and may relate to conducting radiological assessments, patient intake, patient scheduling, billing, generating electronic reports, or other operations of a radiology center. Metrics used to calculate the score may relate to one or more “journeys” experienced by radiology center staff, HCPs, patients, and other stakeholders.

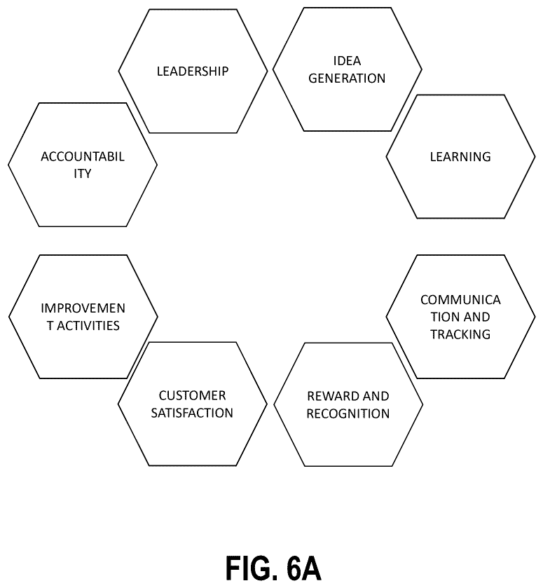

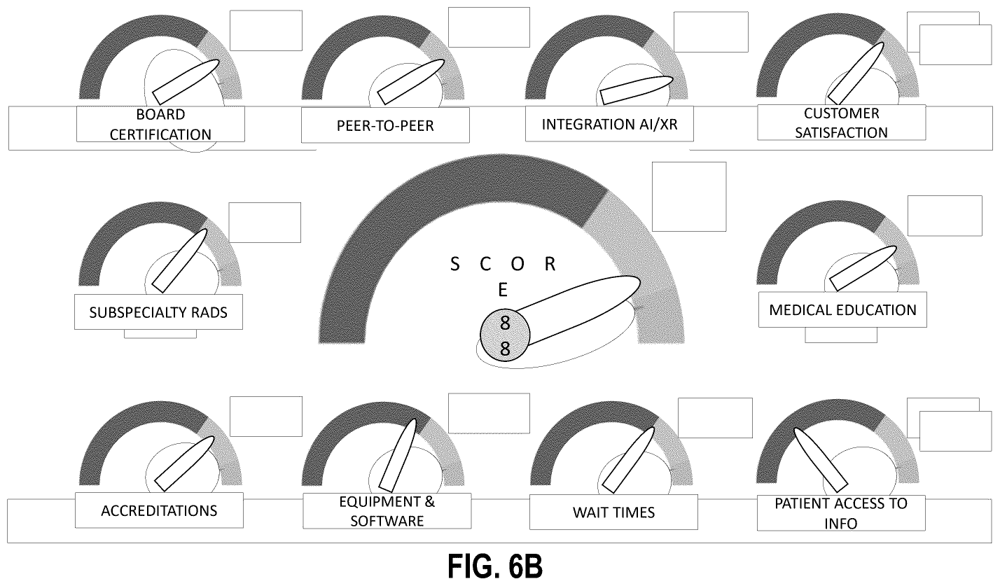

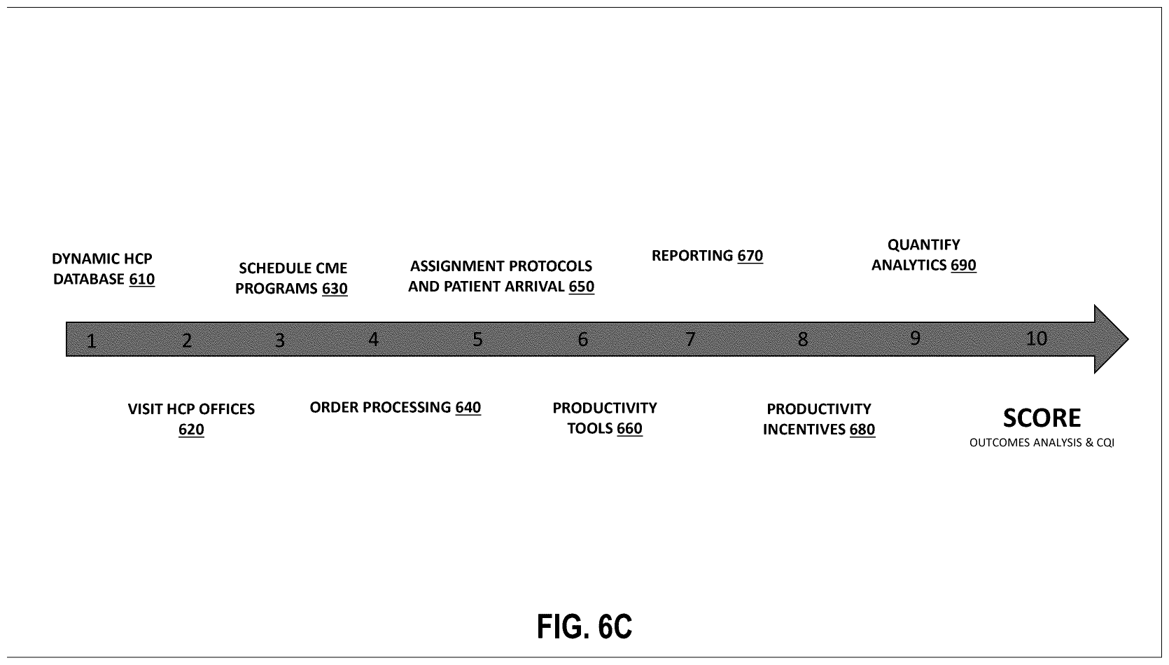

FIGS. 6A-6C illustrate various aspects of the user journey and relate them to a scoring system for radiology centers.

Specifically, FIG. 6A illustrates a set of qualities of radiology centers that the score is configured to assess. For example, a score may reflect patient satisfaction, staff accountability, innovation, dissemination of information, effective leadership, staff performance, data handling and communication, and training opportunities.

FIG. 6B illustrates a set of factors that influence the score. For example, a score may be influenced by types and prevalence of certifications of radiology center staff, peer-to-peer evaluations, integration of technology (e.g., artificial intelligence or augmented reality), patient satisfaction, subspecializations of radiologists or other health care professionals, opportunities for medical education, accreditations of the center and/or of staff, equipment or software available, or patient experience (e.g., patient access to information or wait times).

A radiology center may rate poorly in one or more of these areas and still receive a high score by receiving high ratings in the remaining areas. Conversely, a radiology center may rate highly in one or more areas and receive a poor overall score. Scenarios that may individually or in combination negatively influence the overall score may comprise smaller proportions of certified staff or staff with certifications that are not appropriate for the patient population they serve, low marks in evaluations, use of outdated or poorly-performing (e.g., buggy) technology systems, poor patient reviews, not providing enough continuing medical education (CME), not being accredited, not having physicians with appropriate subspecialties to adequately serve a patient population, or high wait times and low transparency with respect to patient data. Conversely, scenarios that may individually or in combination positively influence the overall score may comprise high availability of certified staff (with appropriate certifications), excellent integration of new technologies, excellent peer-to-peer and/or patient evaluations, ample opportunities for CME, having physicians with appropriate subspecialties, low wait times, and high transparency when providing data to patients.

FIG. 6C illustrates a user journey 600 during which metrics for a radiology center are collected and then used to generate the score. The following paragraphs describe aspects of user journey 600. In other embodiments, a user journey may comprise additional or fewer steps.

### Continuing Medical Education (CMEs) and HCP Data Collection

Patient care coordinators (PCCs) may populate a server (e.g., using a dynamic HCP database 610, configured to be continually updated) with information about HCPs (e.g., ordering physicians (OPs)) to determine their preferences for receiving communications or generated reports. PCCs may receive this information by visiting the radiology centers (HCP offices) 620 where the HCPs work.

A PCC visit to an HCP office 620 may be required to incorporate continuing medical education (CME) 630 helpful to a specific subspecialty. CME may comprise conducting routine meetings to discern ways to improve being a valuable partner in managing an HCP's patients, delivery of published medical articles appropriate to an HCP's clinical practice, providing examples of salient cases illustrating appropriate utilization and how to best assist (best practices) in management of patients, and updating all radiology center staff to ensure all appropriate parties have necessary access to them. CME presentations may be configured according to the preferences of OP or physician staff, (e.g., in terms of topic, location, time, etc.)

### Software Application

HCPs and radiology center staff may engage with a software application (e.g., a client application) providing access to operations of the radiology center.

For example, an HCP may use the application to perform order processing 640 of a radiological assessment, direct to PCC or call center. The order processing 640 may document circumstances (e.g., who placed the order, what was the order regarding, when was the order placed, from what HCP office or which person was the order placed, and how was the order placed) under which the order was placed. Order processing may associate an assessment with an appropriateness value based on a comorbidity index. The appropriateness value may provide a valuable metric for insurance companies (and Medicare) to determine whether or not an assessment should be approved and paid for. The appropriateness value can be quantified as part of the score.

The software application may include a dynamic scheduling function. For example, a scheduler may attempt same day or next day booking (e.g., not allowing a more than two-day wait) and may periodically check to see whether a patient may be scheduled earlier as openings arise to expedite providing results of assessments to an HCP. The scheduler may be configured to immediately notify a patient that is rescheduled.

### Monitoring of Patient

The intelligent healthcare facility operations tracking and record generating system may control assignment protocols and patient arrival activity (650). For example, the intelligent healthcare facility operations tracking and record generating system may periodically retrieve from servers information necessary for conducting an assessment, such as any necessary labs, prior imaging assessments, and clinical notes related to a patient. The intelligent healthcare facility operations tracking and record generating system may verify and ensure whether enough radiology technologists are qualified and available to conduct an assessment (e.g., perform a scheduled examination). The system may disseminate STAT information to ensure personnel are available to assist with an urgent assessment, and that communication to the HCP in charge of the assessment is not delayed. The intelligent healthcare facility operations tracking and record generating system may disseminate STAT information to ensure that all necessary personnel are available to schedule, perform, read and to report the results of the exam to the appropriate OP or designee.

The intelligent healthcare facility operations tracking and record generating system may provide a patient with a communication (e.g., a text or SMS message, email, phone call, or other notification) regarding a scheduled time of an assessment and other information necessary for the patient to show up prepared for the assessment, including financial information and patient obligations. The communication may also include other information about the center (e.g., a mission statement), names of personnel (e.g., physicians and technologists) conducting the assessment and providing care, information needed to access the radiology center, and other patient-specific information (e.g., special needs, transportation assistance, allergies, or medications taken). The patient may be provided with a web chat or page comprising answers to frequently asked questions (FAQs) related to the assessment.

Communications may be sent to a patient the night before and morning of an assessment, confirming the patient's estimated time of arrival and “check in.” A global positioning system (GPS) may signal a front desk of patient arrival at a radiology center. This indication may prompt a welcome message to be sent to the patient, which may also comprise any other information the patient might need for the assessment. If the patient has not arrived by the appointment time, the intelligent healthcare facility operations tracking and record generating system may send a reminder text to the patient. If there is still no response, the intelligent healthcare facility operations tracking and record generating system may generate an automated call to the patient with instructions and queries needed to reschedule, and may notify the radiology staff and may notify the HCP office. The intelligent healthcare facility operations tracking and record generating system may monitor whether the patient is greeted upon arrival at the radiology center. Staff may also be notified immediately when the patient arrives.

The intelligent healthcare facility operations tracking and record generating system may monitor when the patient is brought to an assessment area by a technologist. The system may then log whether the technologist has verified the clinical information (e.g., whether to obtain access to intravenous (IV) medication or administer pharmaceutical or radiopharmaceutical contrast material), and re-verify a protocol for the assessment. The intelligent healthcare facility operations tracking and record generating system may then monitor whether the patient is taken to a room where the assessment is to be conducted. The intelligent healthcare facility operations tracking and record generating system may monitor whether a technologist logs a demeanor of the patient, (e.g., the ability to cooperate and any salient incidents documented (e.g., claustrophobia, grievances, etc.)) The intelligent healthcare facility operations tracking and record generating system may determine whether radiation safety data is logged (e.g., using as low as reasonably possible (ALARA)) principles). After the assessment, the intelligent healthcare facility operations tracking and record generating system may monitor whether the patient is brought to a restroom or back to the lobby or waiting room. The intelligent healthcare facility operations tracking and record generating system may log whether the patient receives further instructions or whether the patient's questions are answered. The intelligent healthcare facility operations tracking and record generating system may monitor whether a technologist enters any additional information discovered about the patient that is not yet in the patient profile, as well as a total wait time recorded from arrival to departure of the patient.

### Monitoring of Staff

The intelligent healthcare facility operations tracking and record generating system may require radiology center staff to confirm protocols for the assessment, including the appropriateness of the assessment, the priority of the assessment, and whether a case associated with an assessment is triaged to a professional with an appropriate subspecialty.

All radiology staff involved in a patient's journey, from marketing to scheduling to report delivery, may be monitored by the intelligent healthcare facility operations tracking and record generating system, to evaluate their productivity and deliver incentives to the radiology staff. Metrics may include the names of each team member involved with patient care, authorization and billing entered, names of individuals spending extra time with a patient, effectiveness of staff with troubleshooting and problem-solving, electronic report turnaround times, feedback from an HCP or from other radiology center staff, increases or decreases in patient referral patterns.

### Center Operations

The intelligent healthcare facility operations tracking and record generating system may track inventory (e.g., daily or monthly) and may track ordering and/or restocking of items, as well as whether items have scanned in or out by staff (e.g., tracking supplies of contrast material (e.g., dye), tubing, needles, gauze, band aids, warm blankets, etc.) The intelligent healthcare facility operations tracking and record generating system may monitor tracer doses delivered, used, or credited.

### Productivity Tools and Reporting

The patient journey may include, as previously discussed relating to FIGS. 1-3, productivity tools (660) configured to provide radiologists or other HCPs with special or innovative software applications configured to increase accuracy and actionable information needed to interpret an assessment and/or provide care. The patient journey may include collecting additional metrics and/or quantities using the additional applications. An electronic report (670) may be generated from the assessment.

### Productivity Incentives

The intelligent healthcare facility operations tracking and record generating system may provide productivity incentives 680. For example, staff involved in a patient's journey, from marketing to scheduling to report delivery, may be documented and credited for their work to evaluate productivity and deliver incentives. Evaluations may be based on, for example, amount and/or quality of time a team member may spend providing patient care, performing administrative tasks (e.g., billing), and troubleshooting radiology center systems (e.g., electronic systems). The evaluations may also be based on customer or patient feedback.

### Score Factors

To determine the score, the intelligent healthcare facility operations tracking and record generating system may quantify analytics (690) resulting from the activities in the preceding paragraphs. Scores for each patient journey may be averaged to generate a monthly, quarterly, or annual score for a radiology center.

An example scoring system follows. A scorecard may be comprised of incremental points (in parentheses) for each assessment, including;
HCP for the requested assessment must be provided with CME no less every 3 months (10)
Documentation that assessment requested was easy to schedule; whether through PCC, application or call center (5)
Assessment scheduled within two hours of order and patient notified (5)
Assessment to be performed within 1-2 business days (5)
Message sent to patient day before and day of study with necessary instructions and explanations (5)
Tech and/or RAD checks for appropriateness and protocols assessment (5)
Patient greeted within 5 minutes of arrival at center (5).
Patient moved from lobby to start IV or to sub-waiting room within 5 min (5)
STAT level clarified and appropriate personnel notified (10)
Patient information reviewed, updated and assessment explained by technologist and/or RAD (5)
ALARA documented (5)
Patient departs center with transportation confirmed and offered “goodie bag” and/or snack (5)
Assessment triaged to appropriate board-certified subspecialist with all necessary clinical information (including OP clinic notes, prior studies and/or reports) (10)
Correlative outside studies or reports garnered, documented, reviewed by RAD and deliberated/contained within in report (10)
Key images (salient image from assessment with pertinent finding displayed in report) and/or AI tools used and documented in report and QR code and/or link to entire study from report (10)
Peer-to-peer documented (5) including reviewing and commenting on prior imaging/reports included.
Final report signed within 24 hours of assessment (5)
“BONUS” Feedback on report via link or QR code on report on five point scale where ⅘ or greater (8-10 respectively)
* NOTE: a “perfect score” could result in a score up to 115, but would only receive 100.

The following steps may be taken to update the score:
A scorecard is assigned to each and every assessment (e.g., radiology exam) (CT, MRI, PET, Ultrasound, etc) performed measuring each factor and assigning points for a total score for that specific exam.
All exams for that day, week, month, quarter and year and “averaged” so that scores for the entire imaging center can be generated in real time.
While it would be optimal to generate a score for each assessment by the end of each day, delays in image distribution, radiologist reading (interpretation, prior study comparison and dictation/transcription) and report distribution make this nearly impossible (though delays also negatively affect the score).
Low scores (below 80%) for any incremental steps are flagged, PCC notified, action plan reviewed and escalated to center director.
* Positive customer feedback earns additional points for that exam.

Described is an alternative or additional scoring system:
1. Updating an HCP server intended to be used to improve open communication between HCP office and radiology center with a goal for CQI and to serve as a valued partner in the service for their practice. No points.
2. Visiting an office of an HCP associated with a radiology center. The routine delivery of medical literature appropriate for OP practice (including interesting cases, technology updates and CME) with a goal to replace the socialization practices routinely done by marketing representatives at imaging centers.
   Visits documented by PCC geofencing.
   CME delivered with all PCC visits and documented/scanned into the intelligent healthcare facility operations tracking and record generating system.
   Clinical staff generates weekly CME materials for each HCP subspecialty.
   PCC delivers monthly CME materials to each.
   5 points. All or none.
3. Scheduling continuing medical education (CME) programs
   CME is requisite for any or all PCC visits to OP offices including AM coffees, lunches, afternoon coffees and lecture series/dinners off-site and
   PCC must detail and documented in the intelligent healthcare facility operations tracking and record generating system.
   5 points. All or none.

### Assessment Level Points

4. Order processing. Ease of scheduling and time to schedule.
   Limit waits for call-in scheduling by HCP office to 2 min. Phone system tracks.
   Create app for HCP to schedule without having to call-in
   5 points. All or none.
5. Assessment scheduled and verified with patient within 2 hours of initial call or app order (input by scheduler).
   5 points. All or none.
6. Assessment performed within two business days (unless patient no-shows or calls to reschedule)
   5 points. All or none.
7. Technologist and/or radiologist checks for appropriateness and protocols assessment (technologist checks with radiologist and checks box indicating so)
   5 points. All or none.
8. Patient greeted within 5 minutes of arrival at radiology center
   if patient has application, then geofencing determines arrival time and check-in time.
   if no application then front desk can input.
   If no application or front desk, patient will be asked to rate and wait times are included.
   5 points. All or none.
9. Patient moved from lobby to start IV or to sub-waiting room within 5 min (input documented by tech and equated with arrival time)
   5 points. All or none.
10. If assessment ordered STAT, level is clarified, appropriate personnel notified and time stamp ensures on-time delivery.
   STAT 1 Needs to be read as soon as completed and ordering physician called.
   STAT 2 Needs a report same day before COB.
   STAT 3 Needs to generate a report same day with report by next morning.
   STAT 4 level subsequently modified by office or ordering physician (i.e. was routine and now a STAT 1-3 and RAD receives notification).
   BONUS 10 points. All or none.
11. Patient information reviewed, updated and assessment explained by technologist and/or RAD to patient, family or patient representative.
   Input by tech and approved by patient on iPad.
   5 points. All or none.
   ALARA documented by tech.
   Provides radiation exposure data generated by each scanner systems to ensure exposure and or radiation safety techniques incorporated into practice.
   5 points. All or none.
12. Patient departs center with transportation confirmed and offered “goodie bad” and/or snack which is all confirmed and documented by tech or front desk.
   5 points. All or none.
13. Assessment triaged and time-stamped to appropriate board-certified RAD or subspecialist with all necessary clinical information (including OP clinic notes, prior studies and/or reports) and verifies whether routine or STAT.
   10 points. All or none.
14. Correlative outside studies or reports garnered, documented by tech, reviewed by RAD and deliberated/contained within in report.
   10 points. All or none.
15. Key images (salient image from assessment with pertinent finding displayed in report) and/or AI tools used and documented in report and QR code and/or link to entire study from report.
   10 points. All or none.
16. Peer-to-peer documented by RAD, including reviewing and commenting on prior imaging/reports included to determine course of disease.
   5 points. All or none.
17. Final report signed within 24 hours of assessment as tracked by time-stamp from image distribution via PACS to RAD to rendering final report (delivered to OP).
   5 points. All or none.
18. Feedback on final report
   via link or QR code from report
   on five point scale where ⅘ or greater (8-10 respectively)*
   No points for 3 or less

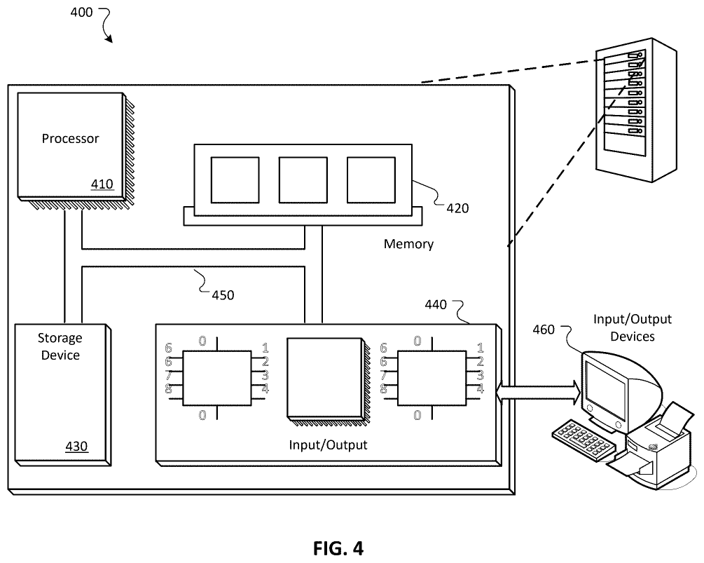

FIG. 4 is a block diagram of an example computer system 400. For example, FIG. 1 could be an example of the system 400 described here, as could a computer system used by any of the users who access resources of FIGS. 1-3. The system 400 includes a processor 410, a memory 420, a storage device 430, and one or more input/output interface devices 440. Each of the components 410, 420, 430, and 440 can be interconnected, for example, using a system bus 450.

The processor 410 is capable of processing instructions for execution within the system 400. The term “execution” as used here refers to a technique in which program code causes a processor to carry out one or more processor instructions. In some implementations, the processor 410 is a single-threaded processor. In some implementations, the processor 410 is a multi-threaded processor. In some implementations, the processor 410 is a quantum computer. The processor 410 is capable of processing instructions stored in the memory 420 or on the storage device 430. The processor 410 may execute operations such as [general topic(s) of application].

The memory 420 stores information within the system 400. In some implementations, the memory 420 is a computer-readable medium. In some implementations, the memory 420 is a volatile memory unit. In some implementations, the memory 420 is a non-volatile memory unit.

The storage device 430 is capable of providing mass storage for the system 400. In some implementations, the storage device 430 is a non-transitory computer-readable medium. In various different implementations, the storage device 430 can include, for example, a hard disk device, an optical disk device, a solid-state drive, a flash drive, magnetic tape, or some other large capacity storage device. In some implementations, the storage device 430 may be a cloud storage device, e.g., a logical storage device including one or more physical storage devices distributed on a network and accessed using a network, such as the network shown in FIG. 1. The input/output interface devices 440 provide input/output operations for the system 400. In some implementations, the input/output interface devices 440 can include one or more of a network interface devices, e.g., an Ethernet interface, a serial communication device, e.g., an RS-232 interface, and/or a wireless interface device, e.g., an 802.11 interface, a 3G wireless modem, a 4G wireless modem, etc. A network interface device allows the system 400 to communicate, for example, transmit and receive data such as radiological image data as shown in FIG. 1, e.g., using the network shown in FIG. 1. In some implementations, the input/output device can include driver devices configured to receive input data and send output data to other input/output devices, e.g., keyboard, printer and display devices 460. In some implementations, mobile computing devices, mobile communication devices, and other devices can be used.

Referring to FIG. 1, the record processing environment components can be realized by instructions that upon execution cause one or more processing devices to carry out the processes and functions described above, for example, processing and standardizing radiological images. Such instructions can include, for example, interpreted instructions such as script instructions, or executable code, or other instructions stored in a computer readable medium.

An intelligent healthcare facility operations tracking and record generating system as shown in FIG. 1 can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

In some examples, the system 400 is contained within a single integrated circuit package. A system 400 of this kind, in which both a processor 410 and one or more other components are contained within a single integrated circuit package and/or fabricated as a single integrated circuit, is sometimes called a microcontroller. In some implementations, the integrated circuit package includes pins that correspond to input/output ports, e.g., that can be used to communicate signals to and from one or more of the input/output interface devices 440.

Although an example processing system has been described in FIG. 4, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as storing, maintaining, and displaying artifacts can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, or a combination of one or more of them.

The term “system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM, DVD-ROM, and Blu-Ray disks. The processor and the memory can be supplemented by, or incorporated in, special purpose logic circuitry. Sometimes a server (e.g., HCP data server as shown in FIG. 4) is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network such as the network shown in FIG. 1. Examples of communication networks include a local area network (“LAN”) and a wide area network (“WAN”), e.g., the Internet.

### Machine Learning

Machine learning models herein may use one or more natural language processing and/or natural language understanding algorithms.

In some embodiments, the machine learning models may be large language models. A large language model (LLM) is a language model may learn statistical relationships from text documents to be able to generate, interpolate, or predict language (e.g., in text form). may be built with a transformer-based architecture. In many cases, LLMs are built using decoder-only architectures.

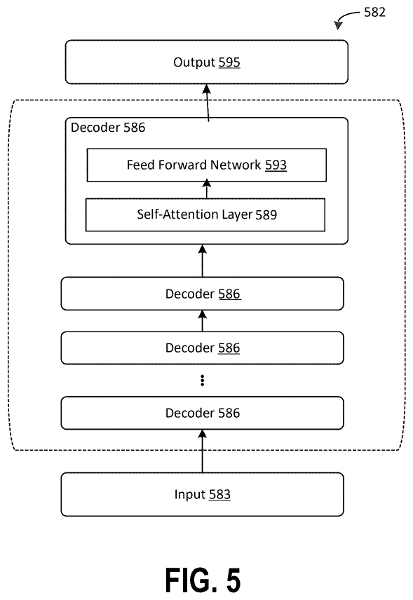

Referring now to FIG. 5, illustrated is a diagram of an implementation of a machine learning model. More specifically, illustrated is a diagram of an implementation of a decoder-only transformer model 582. In some example embodiments, the decoder-only transformer model 582 may implement a machine learning model used to generate the summary electronic report 300. As will be described in more detail, the transformer model 582 may include a self-attention mechanism to capture the relative significance and relationship between different portions of an input 583. For instance, in cases where the input 583 is an image (e.g., a radiological image within an assessment report), the self-attention mechanism of the transformer model 582 may capture the relative significance and relationship amongst different portions (or patches) of the image when generating an output 595 that includes, for example, one or more labels classifying one or more objects present in the image. While the transformer model 582 includes certain features as described herein, these features are provided for the purpose of illustration and are not intended to limit the present disclosure.

As shown in FIG. 5, the transformer model 582 may include a decoder stack having a plurality of decoders 586 (or decoding layers). In the example shown in FIG. 5, the input 583 (e.g., the embedding of each individual portion of the input 583) flows through every decoder 486 in the decoder stack.

Referring again to FIG. 5, the decoder stack may decode the input 583 to generate the output 595, with each decoder 586 in the decoder stack successively decoding the output of the previous decoder 586. For example, the first decoder 586 in the decoder stack may generate a first decoding of the input 583 (e.g., the embedding of each individual portion of the input 583) while the next decoder 586 in the decoder stack may generate a second decoding of the first decoding. As shown in FIG. 5, each decoder 586 may include a self-attention layer 589 and a feed forward network 593. The self-attention layer 589 of the decoder 586 may enable the decoder 586 to generate a context-aware decoding of the input 583 where the decoding for each individual portion of the input 583 incorporates weighted values corresponding to one or more preceding portions of the input 583. For example, in cases where the input 483 is an image, the self-attention layer 485 may determine the relationship between different portions of the image. In some cases, the self-attention layer 589 may include a multi-headed attention mechanism, with each head applying a different set of weights (e.g., query, key, and value weight matrices) for incorporating the other portions of the input 583. It should be appreciated that the weights (e.g., query, key, and value weight matrices) applied by the self-attention layer 589 may be learned during the training of the transformer model 582.

### Tokenization

Transformers convert input text into tokens, which are processed by an encoder and/or decoder. Using a modification of byte-pair encoding, in the first step, all unique characters (including blanks and punctuation marks) are treated as an initial set of n-grams (i.e. initial set of uni-grams). Successively the most frequent pair of adjacent characters is merged into a bi-gram and all instances of the pair are replaced by it. All occurrences of adjacent pairs of (previously merged) n-grams that most frequently occur together are then again merged into even lengthier n-gram repeatedly until a vocabulary of prescribed size is obtained. Token vocabulary consists of integers, spanning from zero up to the size of the token vocabulary. New words can always be interpreted as combinations of the tokens and the initial-set uni-grams.

A token vocabulary based on the frequencies extracted from mainly English corpora uses as few tokens as possible for an average English word.

To find which tokens are relevant to each other within the scope of the context window, the attention mechanism calculates “soft” weights for each token, more precisely for its embedding, by using multiple attention heads, each with its own “relevance” for calculating its own soft weights.

A model may, for example, be pre-trained to predict how the segment continues (autoregressive), or what is missing in the segment, given a segment from its training dataset. An autoregressive model, for example, given a segment “I like to eat”, may predict “ice cream”, or “sushi.” A model trained to predict what is missing, for example, given a segment “I like to [ ] [ ] cream”, may predict that “eat” and “ice” are missing.

Models may be trained on auxiliary tasks which test their understanding of the data distribution, such as Next Sentence Prediction (NSP), in which pairs of sentences are presented and the model must predict whether they appear consecutively in the training corpus. During training, regularization loss is also used to stabilize training. However, regularization loss is usually not used during testing and evaluation.

### Attention

The transformer building blocks are scaled dot-product attention units. For each attention unit, the transformer model learns three weight matrices: the query weights, the key weights, and the value weights. For each token, the input token representation is multiplied with each of the three weight matrices to produce a query vector, a key vector, and a value vector. Attention weights may be calculated using the query and key vectors: the attention weight between two tokens is the dot product between the query and key elements respectively corresponding to each token. The attention weights may be divided by the square root of the dimension of the key vectors, which stabilizes gradients during training, and passed through a softmax which normalizes the weights. The fact that the query weights and key weights are different matrices allows attention to be non-symmetric. The output of the attention unit for a token is the weighted sum of the value vectors of all tokens, weighted by the attention from the token to each other token.

### Multi-Attention

One set of query, key, and value matrices may comprise an attention head. Each layer in a transformer model may have multiple attention heads. Each attention head generates weights signifying attention tokens that are relevant in some way to each token. Using multiple attention heads allow the model to do this for different definitions of “relevance.” Each of many transformer attention heads may each encode different relevance relations that are meaningful to humans. For example, some attention heads can attend mostly to a next word in a sequence, while others mainly attend from verbs to their direct objects. The computations for each attention head can be performed in parallel, which allows for fast processing. The outputs for the attention layer are concatenated to pass into the feed-forward neural network layers.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

### APPENDIX

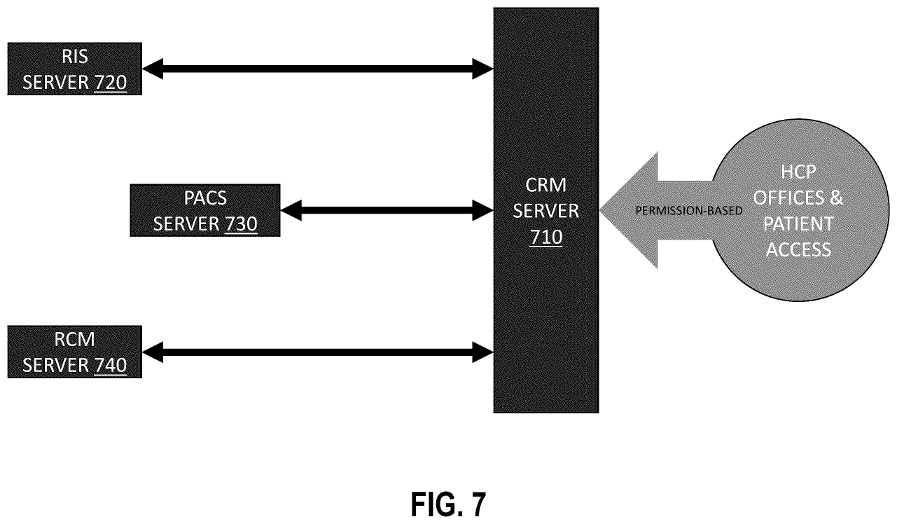

FIGS. 7-14 relate to an additional embodiment of the intelligent healthcare facility operations tracking and record generating system. FIGS. 7-14 and associated description should not be construed to limit any of the preceding disclosure in this specification.

FIG. 7 illustrates an additional embodiment 700 of the intelligent healthcare facility operations tracking and record generating system. Intelligent healthcare facility operations tracking and record generating system comprises a customer relationship management (CRM) server 710, which is communicatively coupled to radiology information system (RIS) server 720, revenue cycle management (RCM) server 740, and picture archiving and communication system (PACS) server 730. RIS server 720 may comprise electronic resources and data for facilitating radiology center operations. PACS server 730 may comprise electronic resources and data for facilitating transfer, storage, and sharing of radiological images. RCM server 740 may comprise electronic resources and data for facilitating medical billing.

CRM server 710 may communicate with PACS server 730, RIS server 720, and RCM server 740 to facilitate, track, and collect data regarding operations within a radiology center, including data gathering, CME, patient intake, patient care, conduct of assessments, and generation of electronic reports.

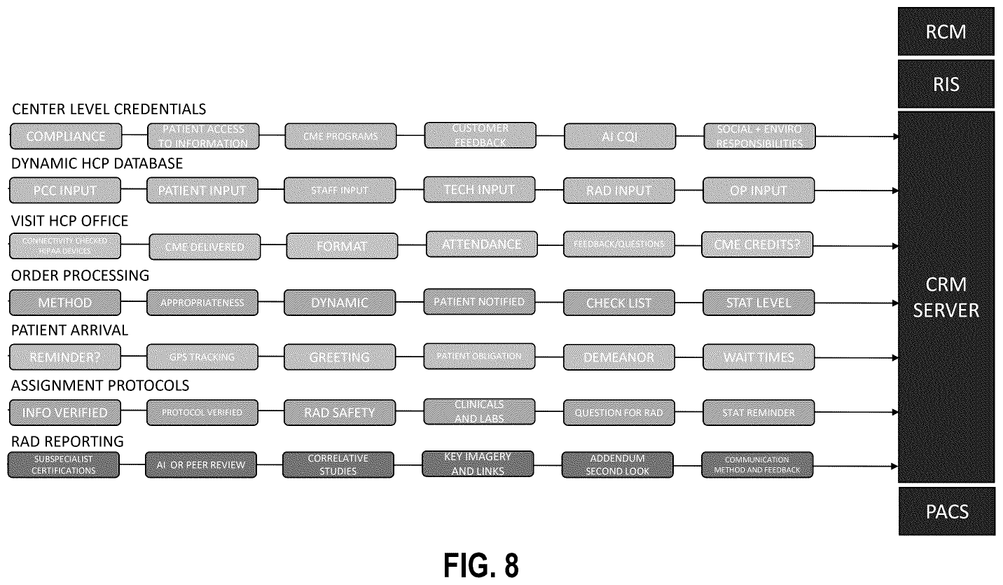

FIG. 8 illustrates an overview of events tracked by CRM server 710, in accordance with some implementations. The events are related to collecting data on HCPs, visits to an HCP office, order processing, patient arrival, assignment protocols, and reporting by radiologists.

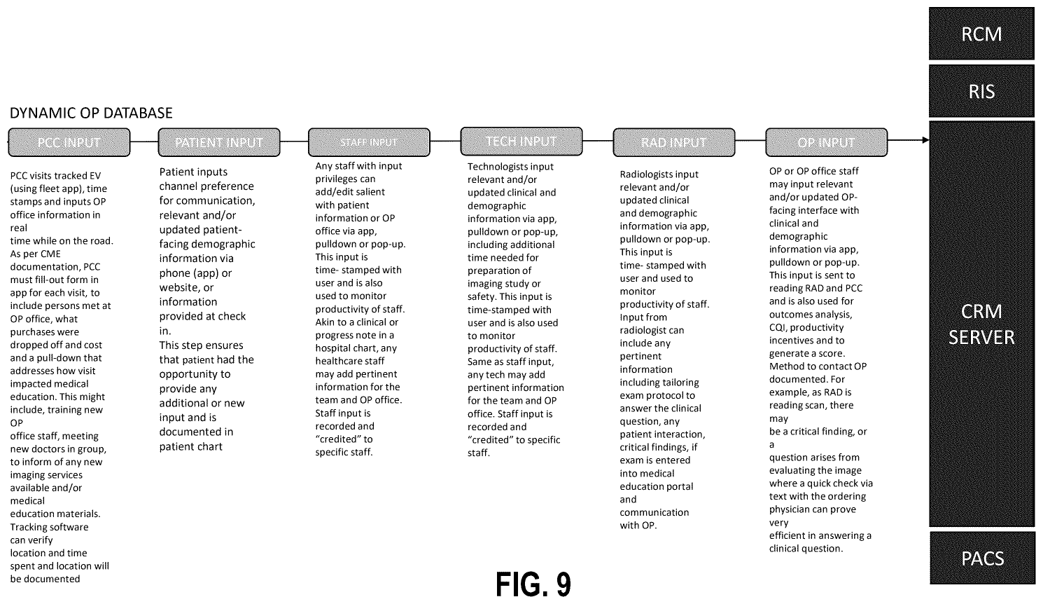

FIG. 9 describes events that are tracked by CRM server 710 related to collecting data on HCPs, in accordance with some implementations.

A PCC visits an OP office using a tracked (e.g., using a fleet app) electric vehicle, time stamps and inputs OP office information in real-time while on the road. As per CME documentation, PCC fills out a form (e.g., using an application) for each visit, including information such as persons met at OP office, what purchases were dropped off and cost, and how the PCC visit impacted medical education (e.g., training new OP office staff, meeting new doctors in group, informing of any new imaging services available and/or medical education materials). Tracking software can verify the PCC location and time spent and location will be documented. A patient can input channel preference for communication, relevant and/or updated patient-facing demographic information via phone (application) or website, or information provided at check-in. This step ensures that the patient had the opportunity to provide any additional or new input and the input is documented in patient chart. Any staff with input privileges can add/edit salient with patient information or OP office via application, (e.g., using a user interface element such as a pulldown or pop-up menu). This input is time-stamped and labeled with the name of the user generating the input and thus can be used to monitor productivity of staff. Akin to a clinical or progress note in a hospital chart, any healthcare staff may add pertinent information for the team and OP office. Staff input is recorded and “credited” to specific staff. Technologists input relevant and/or updated clinical and demographic information via application, pulldown or pop-up, including additional time needed for preparation of imaging study or safety. This input is time-stamped and labeled with the name of the user generating the input and thus can be used to monitor productivity of staff. Same as staff input, any technician may add pertinent information for the team and OP office. Staff input is recorded and “credited” to specific staff. Radiologists input relevant and/or updated clinical and demographic information via application, pulldown or pop-up. T This input is time-stamped and labeled with the name of the user generating the input and thus can be used to monitor productivity of staff. Input from a radiologist can include any pertinent information including tailoring exam protocol to answer the clinical question, any patient interaction, critical findings, if exam is entered into medical education portal and communication with OP.

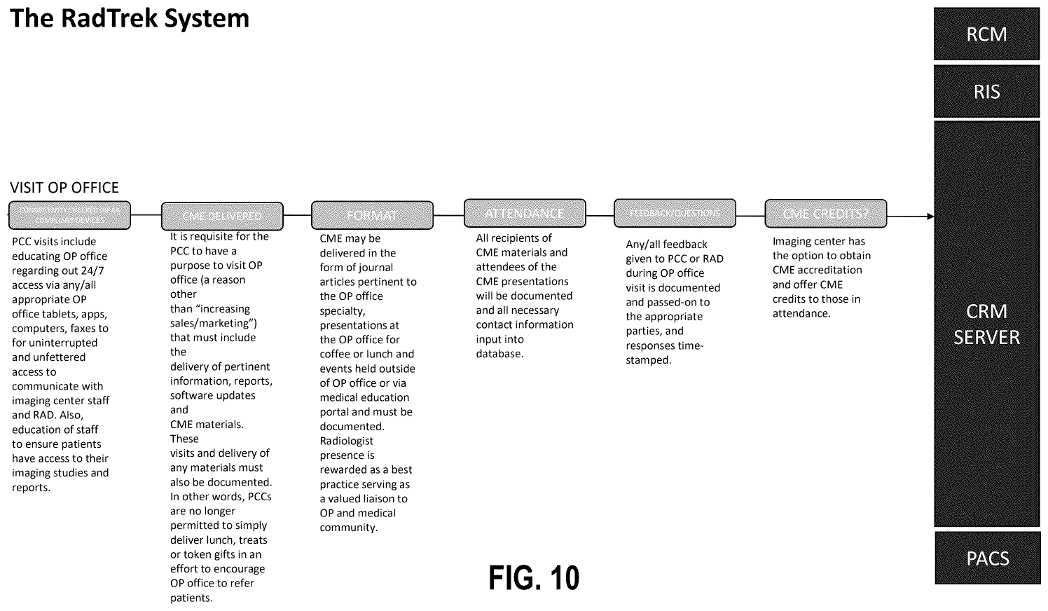

FIG. 10 describes events that are tracked by CRM server 710 related to visits to an HCP office.

PCC visits include educating OP office regarding 24/7 access via any/all appropriate OP office tablets, applications, computers, or faxes to/for uninterrupted and unfettered access to communicate with imaging center staff and RAD. Also, education of staff to ensure patients have access to their imaging studies and reports. It is requisite for the PCC to have a purpose to visit OP office (a reason other than “increasing sales/marketing”) that includes the delivery of pertinent information, reports, software updates and CME materials. These visits and delivery of any materials must also be documented. In other words, PCCs are no longer permitted to simply deliver lunch, treats or token gifts in an effort to encourage OP office to refer patients. CME may be delivered in the form of journal articles pertinent to the OP office specialty, presentations at the OP office for coffee or lunch and events held outside of OP office or via medical education portal and must be documented. Radiologist presence is rewarded as a best practice serving as a valued liaison to OP and medical community. All recipients of CME materials and attendees of the CME presentations will be documented and all necessary contact information input into database. Any/all feedback given to PCC or RAD during OP office visit is documented and passed-on to the appropriate parties, and responses time-stamped. An imaging center has the option to obtain CME accreditation and offer CME credits to those in attendance.

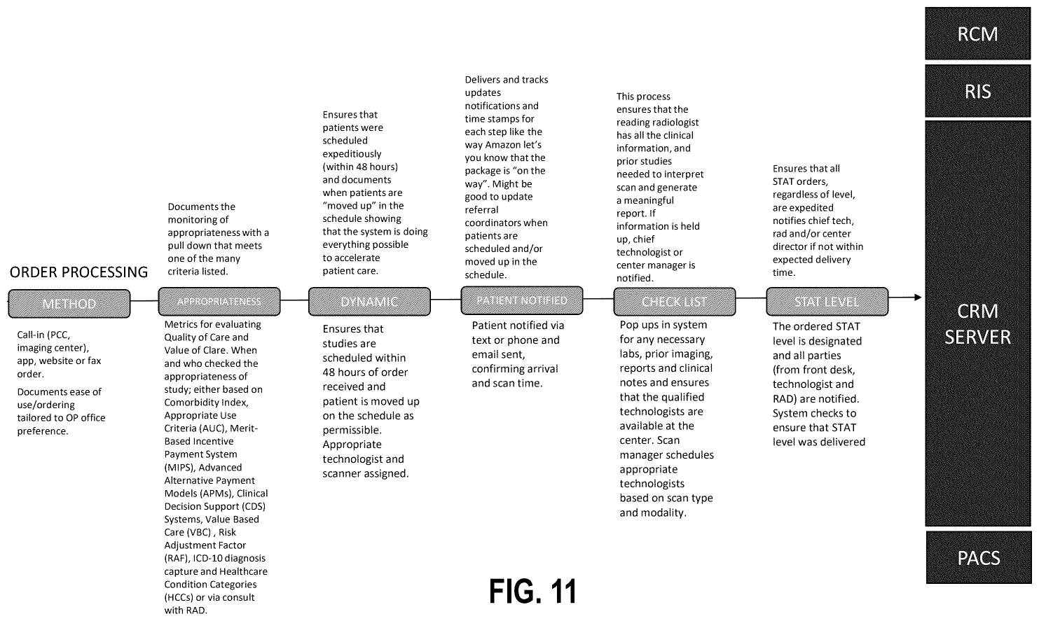

FIG. 11 describes events that are tracked by CRM server 710 related to order processing.

Call-in (PCC, imaging center), app, website or fax order. Documents ease of use/ordering tailored to OP office preference. Documents the monitoring of appropriateness with a pull-down that meets one of the many criteria listed. Metrics for evaluating quality of care and value of Care. When and who checked the appropriateness of study; either based on Comorbidity Index, Appropriate Use Criteria (AUC), Merit-Based Incentive Payment System (MIPS), Advanced Alternative Payment Models (APMs), Clinical Decision Support (CDS) Systems, Value Based Care (VBC), Risk Adjustment Factor (RAF), ICD-10 diagnosis capture and Healthcare Condition Categories (HCCs) or via consult with RAD. Ensures that patients were scheduled expeditiously (within 48 hours) and documents when patients are “moved up” in the schedule showing that the system is doing everything possible to accelerate patient care. Ensures that studies are scheduled within 48 hours of order received and patient is moved up on the schedule as permissible. Appropriate technologist and scanner assigned. Delivers and tracks updates, notifications, and time stamps for each step. Patient notified via text or phone and email sent, confirming arrival and scan time. This process ensures that the reading radiologist has all the clinical information, and prior studies needed to interpret scan and generate a meaningful report. If information is held up, chief technologist or center manager is notified. Pop ups in system for any necessary labs, prior imaging, reports and clinical notes and ensures that the qualified technologists are available at the center. Scan manager schedules appropriate technologists based on scan type and modality.

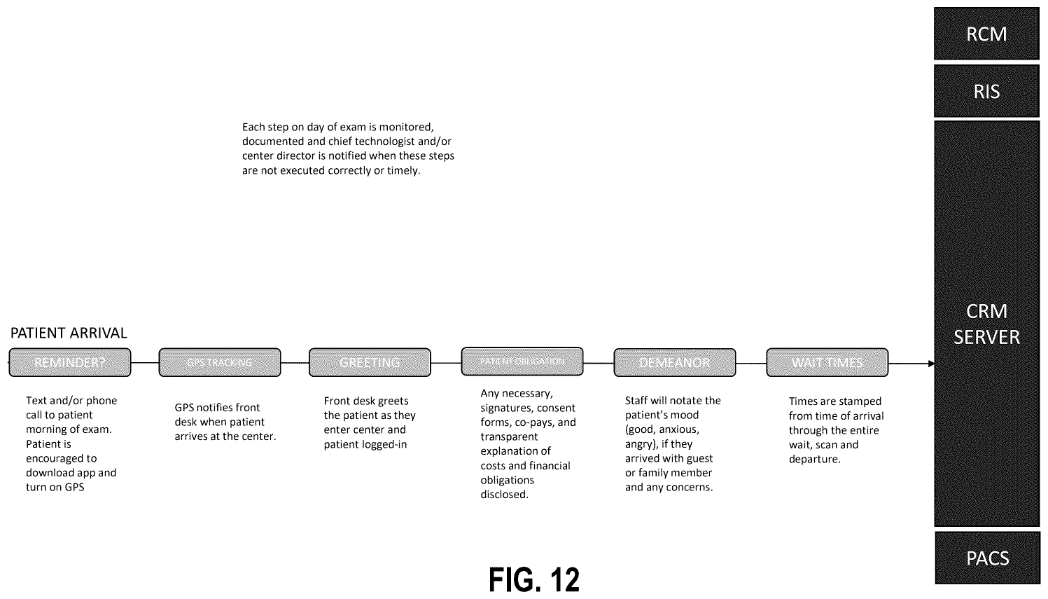

FIG. 12 describes events that are tracked by CRM server 710 related to patient arrival. A text and/or phone call is sent to patient morning of exam.

Text and/or phone call to patient morning of exam. Patient is encouraged to download app and turn on GPS. GPS notifies front desk when patient arrives at the center. Front desk greets the patient as they enter center and patient logged-in. Any necessary, signatures, consent forms, co-pays, and transparent explanation of costs and financial obligations disclosed. Staff will notate the patient's mood (good, anxious, angry), if they arrived with guest or family member and any concerns. Times are stamped from time of arrival through the entire wait, scan and departure. Each step on day of exam is monitored, documented and chief technologist and/or center director is notified when these steps are not executed correctly or timely.

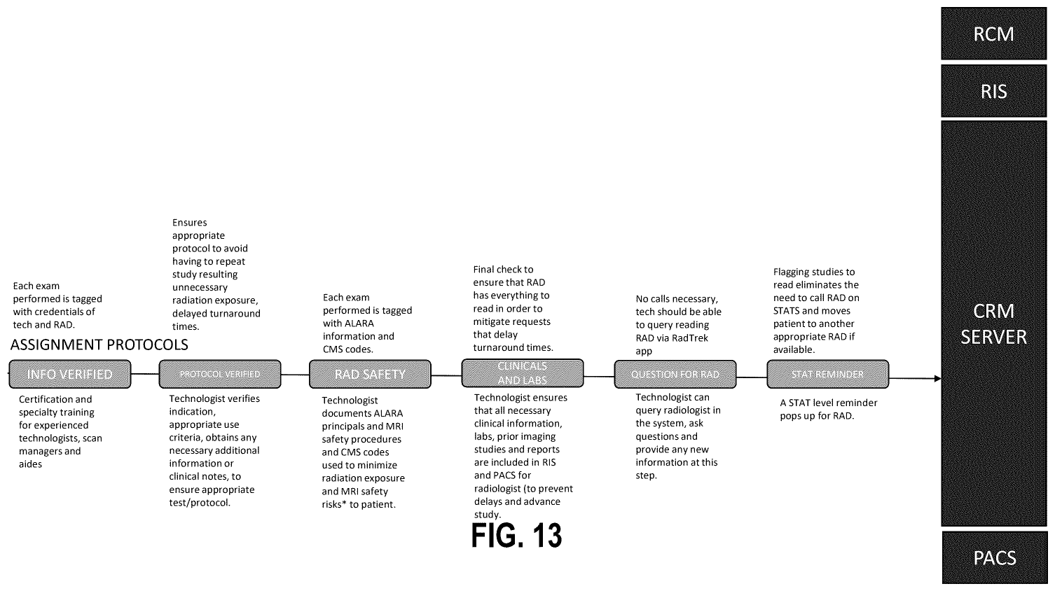

FIG. 13 describes events that are tracked by CRM server 710 related to conduct of assessments.

Certification and specialty training for experienced technologists, scan managers and aides. Each exam performed is tagged with credentials of tech and RAD. Technologist verifies indication, appropriate use criteria, obtains any necessary additional information or clinical notes, to ensure appropriate test/protocol. Ensures appropriate protocol to avoid having to repeat study resulting unnecessary radiation exposure, delayed turnaround times. Technologist documents ALARA principals and MRI safety procedures and CMS codes used to minimize radiation exposure and MRI safety risks to patient. Each exam performed is tagged with ALARA information and CMS codes. Technologist can query radiologist in the system, ask questions and provide any new information at this step. No calls necessary, tech should be able to query reading RAD. A STAT level reminder pops up for RAD. Flagging studies to read eliminates the need to call RAD on STATS and moves patient to another appropriate RAD if available.

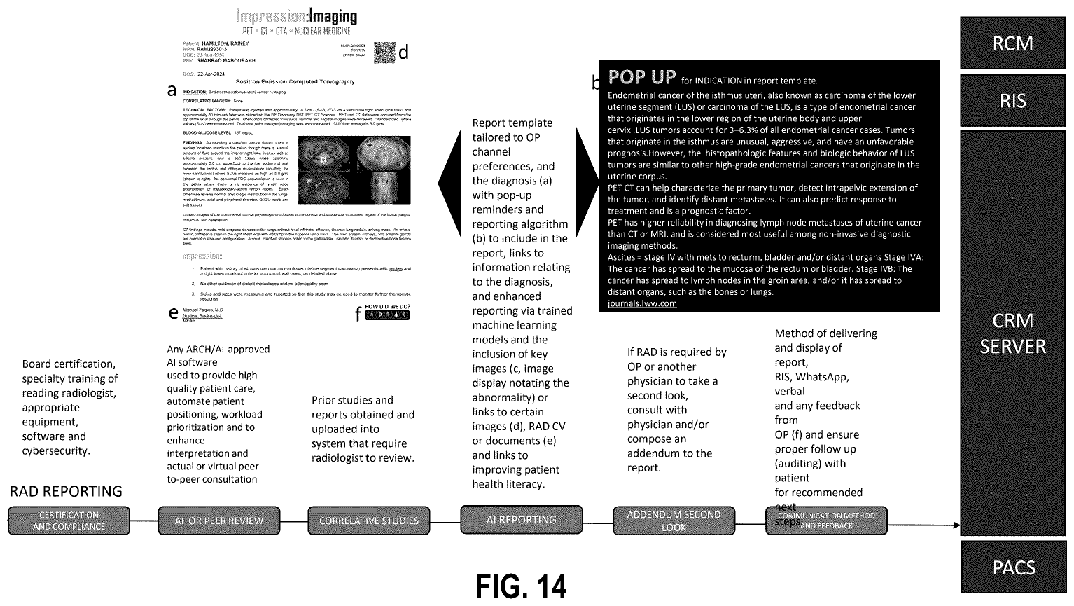

FIG. 14 describes events that are tracked by CRM server 710 related to generation of electronic reports. Board certification, specialty training of reading radiologist, appropriate equipment, software and cybersecurity. Any ARCH/AI-approved AI software used to provide high-quality patient care, automate patient positioning, workload prioritization and to enhance interpretation and actual or virtual peer-to-peer consultation. Prior studies and reports obtained and uploaded into system that require radiologist to review. Report template tailored to OP channel preferences, and the diagnosis (a) with pop-up reminders and reporting algorithm (b) to include in the report, links to information relating to the diagnosis, and enhanced reporting via trained machine learning models and the inclusion of key images (c), image display notating the abnormality or links to certain images (d), RAD CV or documents (e) and links to improving patient health literacy. If RAD is required by OP or another physician to take a second look, consult with physician and/or compose an addendum to the report. Method of delivering and display of report, RIS, text message, verbal and any feedback from OP (f) and ensure proper follow up (auditing) with patient for recommended next steps.

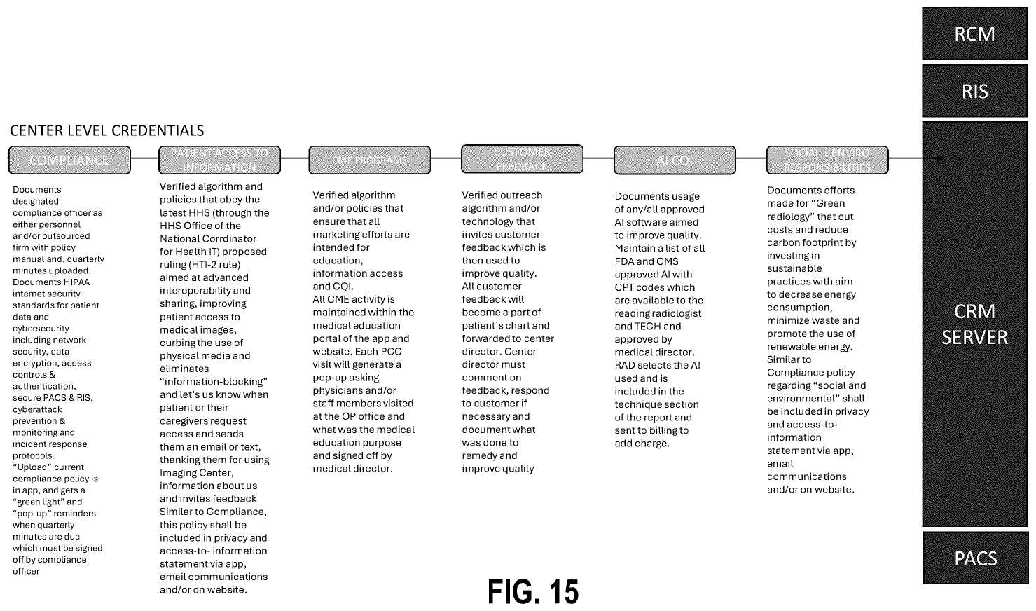

FIG. 15 describes events that are tracked by CRM server 710 related to center level credentials, in accordance with some embodiments. Documents designated compliance officer as either personnel and/or outsourced firm with policy manual and, quarterly minutes uploaded. Documents HIPAA internet security standards for patient data and cybersecurity including network security, data encryption, access controls & authentication, secure PACS & RIS, cyberattack prevention & monitoring and incident response protocols. “Upload” current compliance policy is in application, and gets a “green light” and “pop-up” reminders when quarterly minutes are due which must be signed off by compliance officer. Verified algorithm and policies that obey the latest HHS (through the HHS Office of the National Coordinator for Health IT) proposed ruling (HTI-2 rule) aimed at advanced interoperability and sharing, improving patient access to medical images, curbing the use of physical media and eliminates “information-blocking” and lets us know when patient or their caregivers request access and sends them an email or text, thanking them for using imaging center, information about the system and invites feedback. Similar to Compliance, this policy shall be included in privacy and access-to-information statement via application, email communications and/or on website. Verified algorithm and/or policies that ensure that all marketing efforts are intended for education, information access and CQI. All CME activity is maintained within the medical education portal of the application and website. Each PCC visit will generate a pop-up asking physicians and/or staff members visited at the OP office and what was the medical education purpose and signed off by medical director. Verified outreach algorithm and/or technology that invites customer feedback which is then used to improve quality. All customer feedback will become a part of patient's chart and forwarded to center director. Center director must comment on feedback, respond to customer if necessary and document what was done to remedy and improve quality. Documents usage of any/all approved AI software aimed to improve quality. Maintain a list of all FDA and CMS approved AI with CPT codes which are available to the reading radiologist and TECH and approved by medical director. RAD selects the AI used and is included in the technique section of the report and sent to billing to add charge. Documents efforts made for “green radiology” that cut costs and reduce carbon footprint by investing in sustainable practices with aim to decrease energy consumption, minimize waste and promote the use of renewable energy. Similar to Compliance policy regarding “social and environmental” shall be included in privacy and access-to-information statement via app, email communications and/or on website.

What is claimed is:

1. A method, comprising:
receiving a template electronic report configured to display information in a graphical format associated with one or more preferences of a physician;
receiving patient information associated with a medical assessment conducted on a patient of the physician;
generating a summary electronic report by populating the template electronic report at least in part by processing the patient information and the one or more preferences of the physician with a trained machine learning model;
wherein the summary electronic report is configured to display a summary of the medical assessment in the graphical format associated with the one or more preferences of the physician;
wherein the summary of the medical assessment comprises a subset of information comprised within a complete electronic record of the medical assessment; and
wherein the subset of information comprises at least one image.

2. The method of claim 1, further comprising generating a score for a radiological facility based at least in part on the summary of the medical assessment.

3. The method of claim 1, wherein the patient information comprises a note generated by the physician.

4. The method of claim 3, wherein the note is a handwritten record.

5. The method of claim 1, wherein the summary electronic report comprises one or more vital statistics.

6. The method of claim 1, wherein the summary electronic report comprises one or more treatments or medications administered to the patient during the medical assessment.

7. The method of claim 1, wherein the summary electronic report comprises demographic or medical information of the patient.

8. The method of claim 1, wherein the trained machine learning model comprises a natural language processing model.

9. The method of claim 1, wherein the trained machine learning model comprises a transformer.

10. The method of claim 1, wherein the trained machine learning model comprises a generative pre-trained transformer model.

11. The method of claim 1, wherein the trained machine learning model comprises a neural network.

12. The method of claim 1, wherein the medical assessment is a radiological assessment.

13. The method of claim 1, wherein the at least one image is a radiological image.

14. The method of claim 1, wherein the summary electronic report comprises a visual code configured to, when digitally scanned, access a complete electronic record of the medical assessment.

15. The method of claim 14, wherein the visual code is a quick reference (QR) code.

16. A system, comprising:
a radiology information system (RIS) configured to be implemented by one or more processors;
an image server configured to be implemented by one or more processors; and
a client device, configured to be implemented by one or more processors;
wherein the client device is configured to provide, to the RIS, a template electronic report configured to display information in a graphical format associated with one or more preferences of a physician to the RIS and patient information associated with a medical assessment;
wherein the image server is configured to provide, to the RIS, a radiological image;
wherein the RIS is configured to:
generate a summary electronic report by populating the template electronic report at least in part by processing the patient information and the one or more preferences of the physician with a trained machine learning model;
wherein the summary electronic report is configured to display a summary of the medical assessment in the graphical format associated with the one or more preferences of the physician;
wherein the summary of the medical assessment comprises a subset of information comprised within a complete electronic record of the medical assessment;
wherein the subset of information comprises the radiological image.
